(19) United States
(12) Patent Application Publication
(10) Pub. No.: US 2023/0277869 A1
(43) Pub. Date: Sep. 7, 2023

(54) RADIOTHERAPEUTIC BANDAGE COMPOSITION AND METHOD

(71) Applicant: DB THEREAPEUTICS, INC, Rochester, NY (US)

(72) Inventor: ANTHONY JOSEPH DI PASQUA, Rocheseter, NY (US)

(73) Assignee: DB THEREAPEUTICS, INC, Rochester, NY (US)

(21) Appl. No.: 18/178,500

(22) Filed: Mar. 4, 2023

Related U.S. Application Data
(60) Provisional application No. 63/268,896, filed on Mar. 4, 2022.

(51) Int. Cl. A61N 5/10; B82Y 5/00; A61F 13/02

(52) U.S. Cl. CPC A61N 5/1029; A61F 13/0246; B82Y 5/00

(57) ABSTRACT

A bandage having an electrospun sheet having a polyacrylonitrile nanofiber embedded with a carrier nanoparticle or a carrier particle, the carrier nanoparticle or carrier particle including an activatable nuclide selected from the group consisting of yttrium-89, lanthanum-139, praseodymium-141, samarium-152, dysprosium-164, holmium-165, rhenium-185, rhenium-187, and combinations thereof. The bandage has a laminate enclosure, enclosing the electrospun sheet and the bandage has a distribution of the carrier nanoparticle or the carrier particle to emit a uniform radiation across the surface area of the bandage after neutron-activation.

### CROSS REFERENCE TO RELATED APPLICATIONS

This application claims priority under 35 U.S.C. §119 to U.S. Provisional Application No. 63/268,896, filed Mar. 4, 2022, which is herein incorporated by reference in its entirety.

### TECHNICAL FIELD

This invention relates to radiotherapeutic bandages. More specifically, this invention relates to radiotherapeutic bandages and the treatment of skin cancer and skin diseases using electrospun radiotherapeutic bandages.

### BACKGROUND OF THE INVENTION

Approximately one in five Americans will be diagnosed with skin cancer in their lifetime. The most common form of skin cancer is non-melanoma skin cancer (NMSC), and incidents of NMSC are expected increase by as much as 50% by 2030. This increase is a result of a rise in exposure to ultraviolet light (UV). Most NMSCs arise in body parts that are highly sun-exposed, such as the face, the back of the neck, and the hands.

The most common treatment method for removing localized skin lesions is Mohs micrographic surgery (MMS). MMS excises a lesion, skin layer by skin layer, until all signs of the lesion are removed. Radiation therapy is another method used for treatment. There are two major types of radiation therapy used against NMSCs: External beam radiation therapy and brachytherapy. Both require expensive and specialized machines and for the patients to remain immobile during treatment.

Similar treatments apply to other types of diseases, infections, and cancers.

The present invention provides a solution that does not require surgery or specialized equipment. The present invention also allows the patient to remain mobile during treatment.

### SUMMARY OF THE INVENTION

The present disclosure is directed toward radiotherapeutic bandages, the composition of such radiotherapeutic bandages, and the method of making such radiotherapeutic bandages. This invention further relates to radiotherapeutic bandages and the treatment of skin cancer and skin diseases using electrospun radiotherapeutic bandages.

In one aspect of the present disclosure provided herein is a bandage having an electrospun sheet having a polyacrylonitrile nanofiber embedded with a carrier nanoparticle or a carrier particle, the carrier nanoparticle or carrier particle including an activatable nuclide selected from the group consisting of yttrium-89, lanthanum-139, praseodymium-141, samarium-152, dysprosium-164, holmium-165, rhenium-185, rhenium-187, and combinations thereof. The bandage has a laminate enclosure, enclosing the electrospun sheet and the bandage has a distribution of the carrier nanoparticle or the carrier particle to emit a uniform radiation across the surface area of the bandage after neutron-activation.

In another aspect of the present disclosure provided herein is a bandage having an electrospun sheet having a polyacrylonitrile nanofiber embedded with an iron garnet nanoparticle or an iron garnet particle including an activatable nuclide selected from the group consisting of yttrium-89, lanthanum-139, praseodymium-141, samarium-152, dysprosium-164, holmium-165, rhenium-185, rhenium-187, and combinations thereof. The bandage has a laminate enclosure, enclosing the electrospun sheet and the bandage has a distribution of the iron garnet nanoparticle or the iron garnet particle to emit a uniform radiation across the surface area of the bandage after neutron-activation.

In another aspect of the present disclosure provided herein is a method including: preparing carrier nanoparticles or carrier particles comprising iron garnet and an activatable nuclide selected from the group consisting of yttrium-89, lanthanum-139, praseodymium-141, samarium-152, dysprosium-164, holmium-165, rhenium-185, rhenium-187, and combinations thereof; drying and annealing the preparation; grinding the preparation; stirring and heating dimethylformamide (DMF) and polyacrylonitrile (PAN) and forming a concentration of 10% -20% weight/volume PAN/DMF; mixing the preparation and DMF to form a suspension; sonicating the suspension in an ice bath; adding PAN/DMF to the suspension; mixing PAN/DMF and the suspension to form a mixture; adding the mixture to a carriage of an electrospinning instrument; electrospinning the mixture onto a paper substrate and forming a sheet; cutting the sheet into pieces; enclosing and sealing the pieces with a polymer laminate; forming a bandage by cutting or punching the laminated pieces.

These and other objects, features, and advantages of this disclosure will become apparent from the following detailed description of the various aspects of the disclosure taken in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts a table of activatable nuclides for incorporation into particles or nanoparticles, in accordance with one or more embodiments set forth herein;

FIG. 2 depicts the method steps for forming a neutron-activated bandage, in accordance with one or more embodiments set forth herein;

FIG. 3 depicts an XRD of post-horn sonication-HoIG confirming its garnet structure, in accordance with one or more embodiments set forth herein;

FIG. 4A depicts a sheet of ¹⁶⁵Ho-containing electrospun sheet on top of siliconized brown paper., in accordance with one or more embodiments set forth herein;

FIG. 4B depicts a dark field optical imaging of the ¹⁶⁵Ho-containing electrospun sheet of FIG. 4A having a substantially uniform distribution of nanofibers, in accordance with one or more embodiments set forth herein;

FIG. 5 depicts a sheet of bandages, cut from the electrospun sheet of FIG. 4A and laminated, in accordance with one or more embodiments set forth herein;

FIG. 6 depicts a single rectangular bandage cut from the sheet of the bandages of FIG. 5, in accordance with one or more embodiments set forth herein;

FIG. 7A depicts a circular laminated ¹⁶⁵Ho-containing electrospun bandage, in accordance with one or more embodiments set forth herein;

FIG. 7B depicts a second view of the laminated ¹⁶⁵Ho-containing electrospun bandage of FIG. 7A, in accordance with one or more embodiments set forth herein.

### DETAILED DESCRIPTION OF THE INVENTION

Generally stated, disclosed herein are devices, systems, and methods for treatment of skin cancer.

As used herein, the terms “a” or “an” or “the” may refer to one or more than one. For example, “a” marker can mean one marker or a plurality of markers.

As used herein, the term “about,” when used in reference to a measurable value such as an amount of mass, dose, time, temperature, and the like, is meant to encompass variations of the specified amount. Variations may include, for example, as much as ±10% or as little as ±0.1% of the specified amount, including all variations between.

As used herein, the term “activatable nuclide” refers to a non-radioactive nuclide that may be activated to produce a radionuclide. For example, iron garnet nanoparticles and particles are carriers that can be prepared using lanthanide nuclides (e.g., holmium dysprosium, lanthanum, praseodymium, and/or samarium), rare earth nuclides such as yttrium, and/or rhenium nuclides, that become activated by neutron irradiation. Certain embodiments provide for iron garnet nanoparticles and particles as carriers prepared using nuclides of one of the following: yttrium, holmium, dysprosium, lanthanum, praseodymium, samarium, and/or rhenium. The following table provides examples of activatable nuclides and the corresponding radionuclides.

TABLE 1

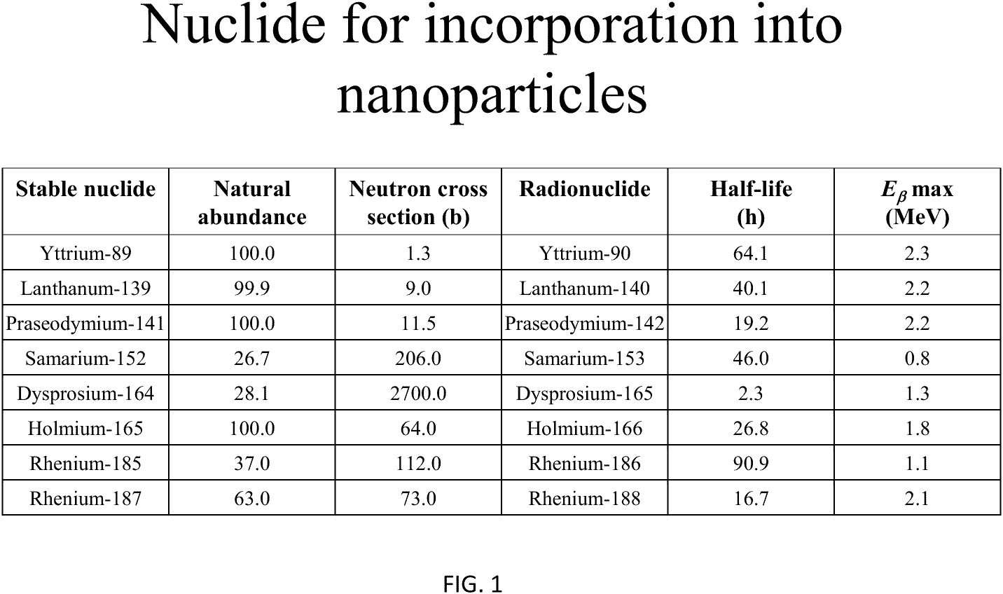

Nuclide for incorporation into nanoparticles (see FIG. 1 for a more detailed version of this table)

| | |
|---|---|
| Activatable nuclide | Radionuclide |
| Yttrium-89 (⁸⁹Y) | Yttrium-90 (⁹⁰Y) |
| Lanthanum-139 (¹³⁹La) | Lanthanum-140 (¹⁴⁰La) |
| Praseodymium-141 (¹⁴¹Pr) | Praseodymium-142 (¹⁴²Pr) |
| Samarium-152 (¹⁵²Sm) | Samarium-153 (¹⁵³Sm) |
| Dysprosium-164 (¹⁶⁴Dy) | Dysprosium-165 (¹⁶⁵Dy) |
| Holmium-165 (¹⁶⁵Ho) | Holmium-166 (¹⁶⁶Ho) |
| Rhenium-185 (¹⁸⁵Re) | Rhenium-186 (¹⁸⁶Re) |
| Rhenium-187 (¹⁸⁷Re) | Rhenium-188 (¹⁸⁸Re) |

In certain other embodiments, doping or incorporating an activatable nuclide into polymeric-based nanoparticles or particles may be performed, with the polymeric-based nanoparticles or particles forming the carriers for the various activatable nuclides listed in FIG. 1. This includes polymeric coating with or encapsulation of the activatable nuclide into the polymeric matrix. In some instances, a chelating agent may be used on the radionuclides for attachment to the polymeric structure. Examples of polymeric-based nanoparticles may include dextran, poly(lactide) (PLA), poly(lactide-co-glycolide) (PLGA) copolymers, poly (ε-caprolactone) (PCL), and poly(amino acids), as well as some natural polymers like alginate, chitosan, gelatin, and albumin.

In certain other embodiments, doping or incorporating activatable nuclides into carbon-based nanoparticles or particles may be performed, with the carbon-based nanoparticles or particles forming the carriers for the various activatable nuclides listed in FIG. 1. Examples of carbon-based nanomaterial or material carriers include but are not limited to: mesoporous carbon nanoparticles (MCNs), carbon nanotubes (CNTs) (including single-walled carbon nanotubes (SWNTs), SWNTs coated with a polydopamine (PDA) shell, and SWNTs coated with a polydopamine (PDA) shell modified by polyethylene glycol (PEG)), fullerenes, carbon dots (CDs), nanodiamonds, and graphene and its derivatives such as graphene oxide nanoplatelets (GONs) (including Non-covalently PEGylated GONs (GONs-PEG)).

In certain other embodiments, doping or incorporating activatable nuclides into lipid-based nanoparticles or particles may be performed, with the lipid-based nanoparticles or particles forming the carriers for the various activatable nuclides listed in FIG. 1.

In certain other embodiments, doping or incorporating activatable nuclides into silica-based nanoparticles or particles may be performed, with the silica-based nanoparticles or particles forming the carriers for the various activatable nuclides listed in FIG. 1. Silica-based nanoparticles may include, for example, mesoporous silica nanoparticles (MSNs), zeolites, or silica metal-organic framework composites.

In still other embodiments, activatable nuclides may, for example, be doped or incorporated into carriers such as quantum dots, gold nanoparticles, silver nanoparticles, rare-earth fluoride nanoparticles, and/or metal oxide nanoparticles.

As used herein, the term “and/or” refers to and encompasses any and all possible combinations of one or more of the associated listed items, as well as the lack of combinations when interpreted in the alternative (“or”).

As used herein, the terms “increase” and “enhance” (and any grammatical variants of these terms) refer to an increase in a specified parameter of at least about 1% to 300% or more, including any and all parameter values therebetween.

As used herein, the terms “inhibit” and “reduce” (and any grammatical variant variants of these terms) refer to a decrease in the specified parameter of at least about 1% to 99% or more, including any and all parameter values therebetween.

As used herein, the term “nanofiber” (and any grammatical variant thereof) refers to a fiber that is about 0.1 nm to about 350 nm in diameter. The term “fiber” (and any grammatical variant thereof) refers to polymer fibers that are between about 0.1 nm and 1 µm in diameter. In some embodiments, the fiber or nanofiber has a diameter of from about 5 nm to about 100 nm or from about 5 nm to about 350 nm. In some embodiments, the fiber or nanofiber is about 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 550, 600, 650, 700, 750, 800, 850, 900, 950, 975 or 999 nm in diameter.

As used herein, the term “nanoparticle” (and any grammatical variant thereof) refers to a particle that is about 0.1 nm to about 250 nm in diameter. The term “particle” (and any grammatical variant thereof) refers to iron garnet particles that are between about 0.1 nm and 1 µm in diameter. In some embodiments, the particle or nanoparticle has a diameter from about 5 nm to about 100 nm or from about 5 nm to about 250 nm. In some embodiments, the particle or nanoparticle is about 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 550, 600, 650, 700, 750, 800, 850, 900, 950, 975 or 999 nm in diameter. Particles and nanoparticles disclosed herein refer to carrier nanoparticles and particles into which particular activatable nuclides are complexed. Carrier particles and nanoparticles may refer to, for example, polymeric-based nanoparticles or particles, carbon-based nanoparticles or particles, lipid-based nanoparticles or particles, silica-based nanoparticles or particles, graphene-based nanoparticles, or other nanoparticles into which particular activatable nuclides are complexed. Particles and nanoparticles disclosed herein may refer to, for example, iron garnet nanoparticles or iron garnet particles into which particular activatable nuclides are incorporated. These carrier particles and nanoparticles may include, for example, yttrium, holmium, lanthanum, praseodymium, samarium, rhenium, and/or dysprosium dispersed within the carrier particles and nanoparticles. Certain embodiments provide for iron garnet nanoparticles or iron garnet particles that contain holmium nuclides. Certain embodiments provide for iron garnet nanoparticles or iron garnet particles that contain activatable nuclides (e.g., yttrium, holmium, dysprosium, lanthanum, praseodymium, samarium, and/or rhenium) dispersed within the iron garnet nanoparticles or iron garnet particles. Certain embodiments provide for carrier nanoparticles and particles may refer to, for example, polymeric-based nanoparticles or particles, lipid-based nanoparticles, silica-based nanoparticles, or other nanoparticles that contain activatable nuclides (e.g., yttrium, holmium, dysprosium, lanthanum, praseodymium, samarium, and/or rhenium) contained within the carrier particles and nanoparticles.

As used herein, “pharmaceutically acceptable” means that the material is suitable for administration to a subject and will allow a desired treatment to be carried out without giving rise to unduly deleterious side effects. The severity of the disease and the necessity of the treatment are generally considered when determining whether any particular side effect is unduly deleterious.

As used herein, the term “radiotherapeutic nanoparticle” refers to a nanoparticle that emits radiation. As used herein, the term “radiotherapeutic particle” refers to a particle or nanoparticle that emits radiation. The term “radionuclide” refers to an atom with an unstable nucleus, which undergoes radioactive decay and emits gamma rays and/or other subatomic particles (e.g., beta particles). A listing of neutron-activatable nuclides for incorporation into particles or nanoparticles is provided in FIG. 1, listing activatable nuclides and their corresponding radionuclides. Examples of activatable nuclides include: ¹⁶⁵Ho, ¹⁶⁴Dy, ⁸⁹Y, ¹³⁹La, ¹⁵²Sm, ¹⁴¹Pr, ¹⁸⁵Re, and ¹⁸⁷Re. Examples of such radionuclides include: ¹⁶⁶Ho, ¹⁶⁵Dy, ⁹⁰Y, ¹⁴⁰La, ¹⁵³Sm, ¹⁴²Pr, ¹⁸⁶Re, and ¹⁸⁸Re. In certain embodiments, the activatable nuclide-containing nanoparticles and/or particles are irradiated/activated with neutron irradiation and result in radiotherapeutic nanoparticles and/or radiotherapeutic particles such that therapeutic levels of radiation are emitted by the radiotherapeutic nanoparticle and/or radiotherapeutic particle. In certain embodiments, the radiotherapeutic nanoparticles and/or radiotherapeutic particles are activated by neutron irradiation such that the nanoparticles or particles emit low “subtherapeutic” levels of radiation, and have little to no effect on cells surrounding the location of the nanoparticles. Subtherapeutic levels of radiation may be, for example, used in conjunction with a radiosensitizer.

As used herein, the term “subject” (and grammatical variants thereof) refers to mammals, avians, reptiles, amphibians, or fish. Mammalian subjects may include, but are not limited to, humans, non-human primates (e.g., monkeys, chimpanzees, baboons, etc.), dogs, cats, mice, hamsters, rats, horses, cows, pigs, rabbits, guinea pigs, ferrets, sheep and goats. Avian subjects may include, but are not limited to, chickens, turkeys, ducks, geese, quail, pheasants, and birds kept as pets (e.g., parakeets, parrots, macaws, cockatoos, and the like). In particular embodiments, the subject is a laboratory animal. Human subjects may include neonates, infants, juveniles, adults, and geriatric subjects. Therapeutic and subtherapeutic variants of embodiments of the invention may be, for example, used upon subjects.

As used herein, the term “therapeutically effective” refers to some improvement or benefit to the subject. Alternatively stated, a “therapeutically effective amount” is an amount that will provide some alleviation, mitigation, or decrease in at least one clinical symptom in the subject (e.g., reduced tumor size, decreased incidence of metastasis, etc. for subjects having a form of cancer). Those skilled in the art will appreciate that the therapeutic effects need not be complete or curative, as long as some therapeutic benefit is provided to the subject. The concentration of stable activatable particles (or nanoparticles) and/or radiotherapeutic agent in the pharmaceutical composition may vary widely (i.e., from less than about 0.05% to about 90% or more by weight) in accordance with the particular mode of administration, the disease(s)/disorder(s)/symptom(s) being treated, the age/weight of the subject, etc. In certain embodiments, the concentration of stable activatable particles or nanoparticles may vary.

As used herein, the terms “treatment,” “treat,” and “treating” refer to providing a subject with the particles and/or nanoparticles disclosed herein in an effort to alleviate, mitigate, or decrease at least one clinical symptom in the subject.

Methods of the present invention may be used to treat any suitable disorder known in the art, including, but not limited to, bacterial infections, viral infections, cancer, trigeminal neuralgia, severe thyroid eye disease, pterygium, pigmented villonodular synovitis, vascular restenosis, heterotopic ossification, rheumatoid arthritis, synovial osteochondromatosis, synovial chondromatosis and hemathrosis. In some embodiments, the disorder is a hematological cancer or cancer. Embodiments of the invention, as understood by one skilled in the art, may be used to treat forms of cancer. As an example, non-melanoma skin cancer may be treated by embodiments of the present invention. It is also understood that one skilled in that art may use embodiments, of the present invention to treat skin diseases. Example of such skin diseases include: actinic keratosis, psoriasis, infantile hemangiomas, among others.

The present invention provides radiotherapeutic nanoparticles and radiotherapeutic particles. In particular embodiments, the present disclosure provides iron garnet nanoparticles or iron garnet particles that contain an activatable nuclide, such as holmium, and/or other activatable nuclides as shown in the table of FIG. 1. Thus, one aspect of the invention provides a stable activatable iron garnet nanoparticle or particle comprising, consisting essentially of or consisting of an activatable nuclide precursor. A further aspect of the invention provides a pharmaceutical composition comprising, consisting essentially of, or consisting of the disclosed iron garnet nanoparticles and/or iron garnet particles and a pharmaceutically acceptable carrier.

An aspect of the invention provides for an electrospun bandage that is impregnated with iron-garnet (commonly referred to as IG) nanoparticles or particles disclosed herein. As used herein, the term “bandage” (and any grammatical variants thereof) refers to a wound, injury, or growth covering into which the disclosed IG nanoparticles or particles have been impregnated. Such bandages may be formed from an electrospun fabric, electrospun patch, electrospun sheet, electrospun mat, or electrospun film. In some embodiments of this aspect of the invention, a bandage is produced by dispersing the disclosed IG nanoparticles or particles and dissolving a polymer in a solvent, applying a stable nuclide solution on a release paper by a coater, and drying. The bandage may then be irradiated with neutrons in a nuclear reactor. Another embodiment of the disclosed invention provides using an electrospinning technique to prepare ¹⁶⁵Ho-loaded nanofibers that can be electrospun into a bandage with uniform nanoparticle or particle distribution. In certain embodiments, nanofibers may be loaded with activatable nuclides other than ¹⁶⁵Ho, such as, for example, ¹⁶⁴Dy, ⁸⁹Y, ¹³⁹La, ¹⁵²Sm, ¹⁴¹Pr, ¹⁸⁵Re, and ¹⁸⁷Re. In the above examples, IG may be a carrier for a nuclide such as, for example, Ho and the other identified activatable nuclides. In certain embodiments, nanoparticles or particles that are used in the manufacture of an electrospun fabric, electrospun patch, electrospun sheet, electrospun mat, or electrospun film can have a size of less than about 500 nm, less than about 400 nm, less than about 300 nm, less than about 200 nm, less than about 100 nm, less than about 50 nm. In certain other embodiments, particles that are used in the manufacture of an electrospun fabric, electrospun patch, electrospun sheet, electrospun mat, or electrospun film can have a size of less than about 400 µm, less than about 200 µm, less than about 100 µm, less than about 50 µm, less than about 20 µm, less than about 10 µm, less than about 5 µm.

In certain other embodiments, carriers other than IG nanoparticles and particles may be used. Doping or incorporating activatable nuclides into polymer nanoparticles and particles may be performed to form the carrier for the various activatable nuclides listed in FIG. 1. Examples of other carriers include but are not limited to mesoporous silica nanoparticles (MSNs), mesoporous carbon nanoparticles (MCNs), carbon nanotubes (CNTs) (including single-walled carbon nanotubes (SWNTs), SWNTs coated with a polydopamine (PDA) shell, and SWNTs coated with a polydopamine (PDA) shell modified by polyethylene glycol (PEG)), and Graphene oxide nanoplatelets (including Non-covalently PEGylated GONs (GONs-PEG)).

The polymer dissolved with the nanoparticles or particles placed in a solvent may include, for example: biodegradable polymers, polylactic acid, polycaprolactone, poly(glycolic acid) (PGA), polydioxanone, polyanhydrides, polyorthoesters, poly(amino acids), chitosans, and sulfonated chitosan. Further polymer examples include: water-soluble polymers such as, poly(vinyl pyrrolidone) (PVP), poly(ethylene oxide) (PEO), poly(vinyl alcohol) (PVA), and poly(acrylic acid) (PAA) and mixtures thereof. Still further polymer examples include: nylon, polyesters, polyamides, poly(amic acids), polyimides, polyethers, polyketones, polyurethanes, polycaprolactones, polyacrylonitriles, polyaramides, polybenzimidazole, poly(2-methoxy, 5 ethyl (2&#39; hexyloxy) para-phenylene vinylene) (MEH-PPV), polyphenylenevinylenes, polyarylene-vinylenes, polythienolene-vinylenes, polypyrrolo-vinylenes, polyheteroarylene-vinylenes, polyanilines, polyphenylenes, polyarylenes, polythiophenes, polypyrroles, polyheteroarylenes, polyphenylene-ethynylenes, polyarylene-ethynylenes, polythieno-ethynylenes, polyheteroarylene-ethynylenes, and mixtures thereof. Other polymers may be used, but it is preferred that the polymer remain stable to at least nuclide activation temperatures.

As used herein, the phrases “relatively uniform distribution of said nanoparticles/particles within said electrospun fabric, electrospun patch, electrospun sheet, electrospun mat, or electrospun film” and “relatively uniform radiation across the surface area of said electrospun fabric, electrospun patch, electrospun sheet, electrospun mat, or electrospun film” relate to the distribution of the nanoparticles/particles and the emitted radiation from said nanoparticles/particles within the electrospun fabric, electrospun patch, electrospun sheet, electrospun mat, or electrospun film. In this regard, the variance with respect to the number of nanoparticles/particles or the amount of emitted radiation for a given surface area of the electrospun fabric, electrospun patch, electrospun sheet, electrospun mat, or electrospun film varies by less than about 30%, less than about 25%, less than about 15%, less than about 10%, less than about 5% or less than about 1%. While the terms electrospun fabric, electrospun patch, electrospun sheet, electrospun mat, and/or electrospun film have been used, these may be used interchangeably.

### Example for Preparation of a Radiotherapeutic Bandage

Nanofiber mats containing ¹⁶⁵Ho were prepared via needleless electrospinning. The input materials and preparation for electrospinning are described as follows. Holmium iron garnet (Ho₃Fe₅O₁₂ or commonly referred to also as HoIG) was produced using the methods described in Munaweera I, Levesque-Bishop D, Shi Y, Di Pasqua AJ, Balkus Jr KJ., Radiotherapeutic bandage based on electrospun polyacrylonitrile containing holmium-166 iron garnet nanoparticles for the treatment of skin cancer. ACS Appl Mater Interfaces; and Koneru B, Shi Y, Munaweera I, Wight-Carter M, Kadara H, Yuan H Di Pasqua AJ, Balkus Jr KJ., Radiotherapeutic bandage for the treatment of squamous cell carcinoma of the skin. Nucl Med Biol. The HoIG was dried and annealed at 950° C. for 4 hours. It was ground lightly via mortar and pestle to break up agglomerates. Dimethylformamide (anhydrous, 99.8%) (DMF) and polyacrylonitrile (MW 150,000) (PAN) were used as received from Millipore Sigma. A 14.29% w/v PAN/DMF (hereby referred to as “conc. PAN/DMF”) was produced through heating and stirring overnight. 1.21 g holmium iron garnet was poured into 3.43 g DMF in a glass vial and vortex mixed at 3000 rpm for 2 minutes. The holmium/DMF suspension was sonicated in an ice bath using a 3 mm diameter probe on a VCX 130 horn sonicator (Sonics and Materials Inc.) at 104.6 kWs/mL power density. After sonication, 8.30 g conc. PAN/DMF was added to the suspension and the mixture vortex mixed at 3000 rpm for 5 minutes. The final mixture was immediately poured into a 10 mL carriage of a NS Lab roll-to-roll electrospinning instrument and spun onto a 50 cm wide siliconized brown paper substrate under the following conditions: Substrate speed: 5 mm/min; Carriage speed: approximately 180 cm/s; Electrode distance: 180 mm; Voltage: 80 kV; Humidity: approximately 18-21%; Temperature: approximately 20-35° C. The mat formed may be, for example, a non-woven fabric. The electrospun mat was nipped at 212° F. (100° C.) for 20 seconds, and 0.5” diameter circles cut with Orion Motor Tech 40 W CO₂ laser cutter using following conditions: 6% laser power and 20 mm/s vector cut.

The electrospun mat may have a distribution of nanofibers and fibers and a distribution of nanoparticles and particles with a range of diameters. For example, nanofibers or fibers in the non-woven fabric forming the mat may have a majority of diameters from about 100 nm to about 350 nm. For example, nanoparticles or particles may have a majority of diameters from about 15 nm to about 250 nm. However, smaller dimensions provide for a better encapsulation of nuclides within the carrier and better encapsulation within the mat. The thickness of the electrospun mat also inversely impacts emission efficiency, with thinner mats having greater emission efficiency.

X-ray powder diffraction (XRD) was used to characterize ¹⁶⁵Ho iron garnet (HoIG) nanoparticles, dark field optical imaging was used to determine the size uniformity of nanofibers in the electrospun mat, and inductively coupled plasma-mass spectrometry (ICP-MS) was used to measure the Ho and iron (Fe) content of the mat, as well as the uniformity of said mat. Six separate areas in three different A4 sheets of the mat were cut, and values obtained.

Pieces were laser cut and laminated with wear-resistant nylon about 25.4 µm (0.001″) thick (McMaster-Carr, Princeton, NJ) and cut again, to ensure complete encapsulation of the holmium-containing nanofibers or fibers, nanoparticles or particles. The wear-resistant nylon used had a tensile strength of approximately 11,200 psi - 12,300 psi (77220 kPa – 84805 kPa). A heat press was preheated to 425° F. (218° C.) (GeoKnight JetPress 14). A siliconized release paper substrate and a single wear-resistant nylon sheet (McMaster-Carr product 8539K199) were placed on the base of the heat press, the 0.5” (12.7 mm) round PAN-HoIG composite pieces were placed in an array on the nylon sheet with approximately 0.75 in (about 19 mm) edge to edge spacing between sample pieces. A second wear-resistant nylon sheet was placed on the opposite side of the pieces, fully enclosing the pieces in the laminate material. The first and second nylon sheets sealed the pieces. A final siliconized release paper layer was placed onto the array of pieces. The spacing between pieces provides for a border around the pieces and provides for a laminated area, which may be used to cut or punch the bandages, while maintaining full sealing of the electrospun PAN-HOIG composite pieces.

The layer setup placed in a heat press unit is release paper – nylon sheet – HoIG pieces –nylon sheet – release paper. The heat press was run at 425° F. (about 218° C.) for approximately 30 seconds and the materials allowed to cool in air. The heat press was run for approximately 30 more seconds.

The laminated array of pieces was allowed to cool to room temperature and was wiped down with Kimwipes. Then, pieces were punched out using a 0.75 in (about 19 mm) die punch and hammer. The electrospun fiber samples were fully encapsulated in the laminate material, maintaining nanofibers and fibers and nanoparticles and particles within. In an alternate embodiment, the samples may be cut into smaller pieces of a desired size and/or shape.

Laminated samples were neutron-activated (or neutron irradiated) for 10 h at 7.5 × 10¹² neutrons/cm²·s and radioactivity quantified at NC State. A high purity germanium detector and associated Canberra MCA gamma spectroscopy system calibrated with a certified mixed-nuclide standard to assay the 1379 keV ¹⁶⁶Ho peak was used. The ¹⁶⁶Ho 1379 keV peak has a lower gamma yield than the 81 keV peak, but the measurement of the detector efficiency at that energy has much less uncertainty than at 81 keV.

In the process described, wear-resistant nylon was used. In certain embodiments of the process other polymer laminates may be used, such as, for example, polyesters, polypropylene, or polyethylene. The laminate may include films or sheets. The laminate heating times may vary with the with the thickness of the laminate and the type of material.

In certain embodiments of the process, laminated samples created in a clean room environment may remove the need to use Kimwipes or similar cleaning tools. While the use of Kimwipes is mentioned, other cleaning tools may also be used.

In some embodiments, the thickness of nylon laminate may be thinner or thicker than 25.4 µm. Nylon that has a thickness of less than 25.4 µm may be used. The thinner the nylon laminating film the greater the permeability of the film to beta particles, in the activated bandages. While thicker nylon laminating film may be used, beta emission may be impeded by the thicker laminating material. At thicknesses suitable for the bandage for skin surface placement, gamma emissions are not significantly impeded. However, beta particle emissions have been determined to be therapeutically effective for diseases like skin cancers, and it is desirable to maximize permeability of the film to beta particles.

The measurements used in the example describe a particular embodiment. However, those skilled in the art would understand that the measurements, and materials may be adjusted to form input materials for the electrospinning process to form desired outputs. Furthermore, measurements, and materials may be adjusted to form input materials for different types and sizes of electrospinning devices with different operating conditions and parameters.

In another example, the drying and annealing temperature of HoIG may range from approximately 800° C. to approximately 1250° C. In yet another example, the length of time for drying and annealing may range from approximately 2 hours to approximately 10 hours.

Standard commercially available Dimethylformamide (anhydrous, 99.8%) (DMF) and polyacrylonitrile (MW 150,000) (PAN) were used. Other concentrations and molecular weights may be used. For example, a lower water content of DMF is preferred, but a higher water concentration may be used. Also, PAN with a lower or higher molecular weight may be used.

In the example, the PAN/DMF concentration was described as 14.29%. However, in other examples concentrations of PAN/DMF may be from, for example, approximately 10% to approximately 20% PAN/DMF may be used.

In the example, 1.21 g HoIG was poured into 3.43 g DMF for mixing to form a suspension. In certain other embodiments, these amounts may be adjusted with the electrospinning device used, the output size of the mat, and the desired concentration of HoIG. The amount of HoIG may be from approximately 1 g - 2 g and the amount of DMF may be from approximately 2.8 g - 5.7 g. Sonication, of the suspension may be performed to inhibit clumping of the particles or nanoparticles and maximize dispersion to form a substantially homogeneous suspension. The sonication time and power density used to form a substantially homogeneous suspension may vary with the sonication device used. While sonication is the preferred method for forming a substantially homogeneous suspension, other standard laboratory devices may be used. Still continuing with the example, 8.3 g of the PAN/DMF was added to the HoIG/DMF mixture. In other examples, the PAN/DMF added may be from approximately 6.8 g - 13.7 g.

While the above process for creating a bandage was described using holmium-165, other activatable nuclides may be used such as, for example, yttrium-89, lanthanum-139, praseodymium-141, samarium-152, dysprosium-164, holmium-165, rhenium-185, rhenium-187, and/or any combination thereof and/or combinations with holmium-165. A carrier such as iron garnet may be combined with activatable nuclides to produce the bandage.

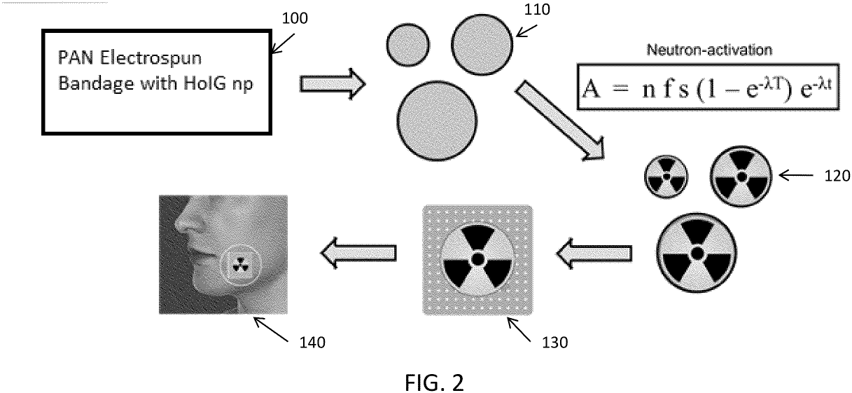

With reference to FIG. 2, a PAN electrospun mat with HoIG nanoparticles is formed 100. The mat may be cut at this point into pieces, for example, strips or circles or squares. The terms cut is used to form smaller pieces of a desired size and/or shape, but these pieces may also be punched into a desired size and/or shape. The HoIG nanoparticles are in a stable nuclide state as distributed within the electrospun bandage 110. While strips or circles or squares are mentioned, the bandage may be cut into pieces of any shape. The cut pieces are laminated and fully sealed. The pieces of electrospun bandage are placed in a reactor where the non-radioactive ¹⁶⁵Ho is made radioactive ¹⁶⁶Ho via neutron-activation 120. After neutron-activation, ¹⁶⁶Ho emits both beta particles and gamma photons upon decay. An adhesive may be, for example, also applied to the activated cut piece forming a bandage for attachment to a patient’s skin 130. The cut piece of activated bandage may be applied to an area of skin needing treatment 140.

In another embodiment, mesoporous silica may be used instead of the iron garnet as carrier of the nuclide.

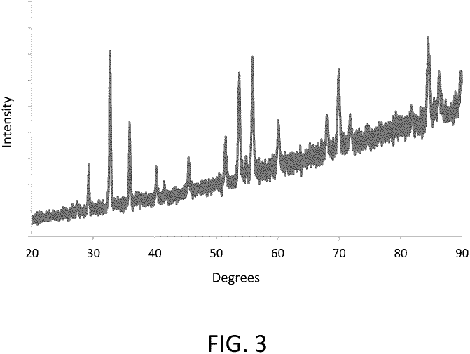

FIG. 3 shows an XRD of post-horn sonication-HoIG and confirms its garnet structure.

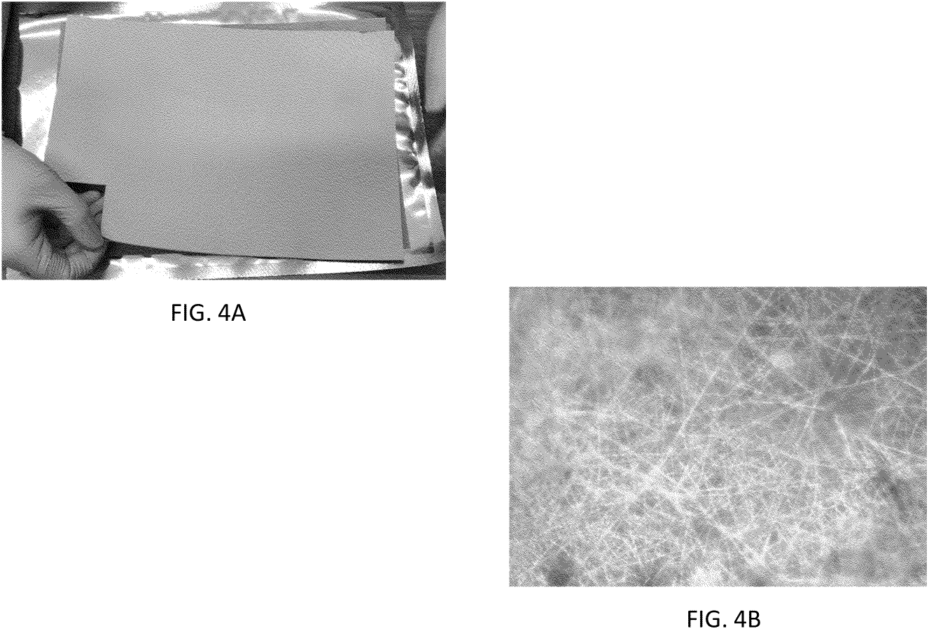

FIG. 4A shows an A4-sized sheet of ¹⁶⁵Ho-containing PAN mat on top of siliconized brown paper. FIG. 4B shows a dark field optical imaging of the ¹⁶⁵Ho-containing mat having a uniform distribution of nanofibers.

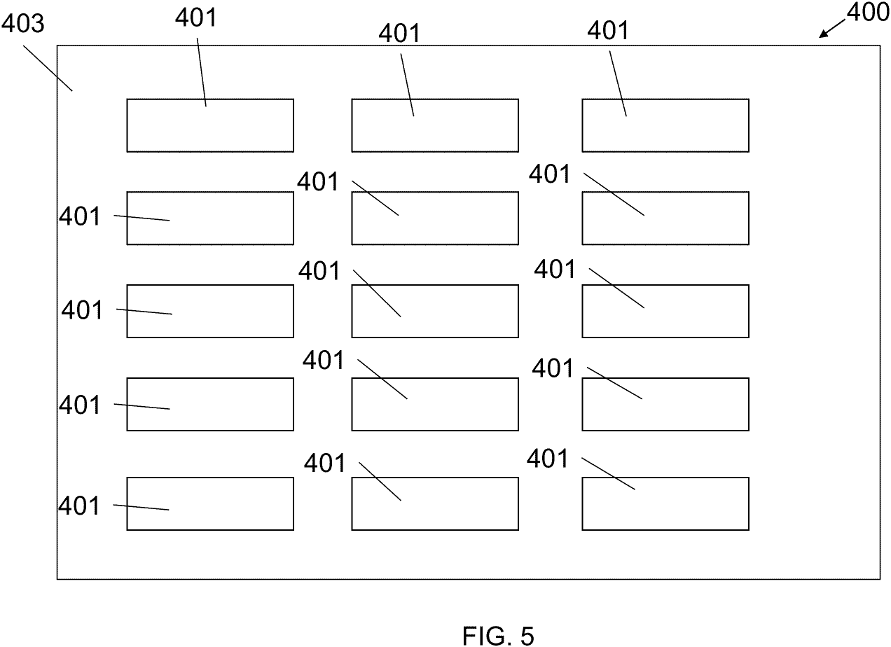

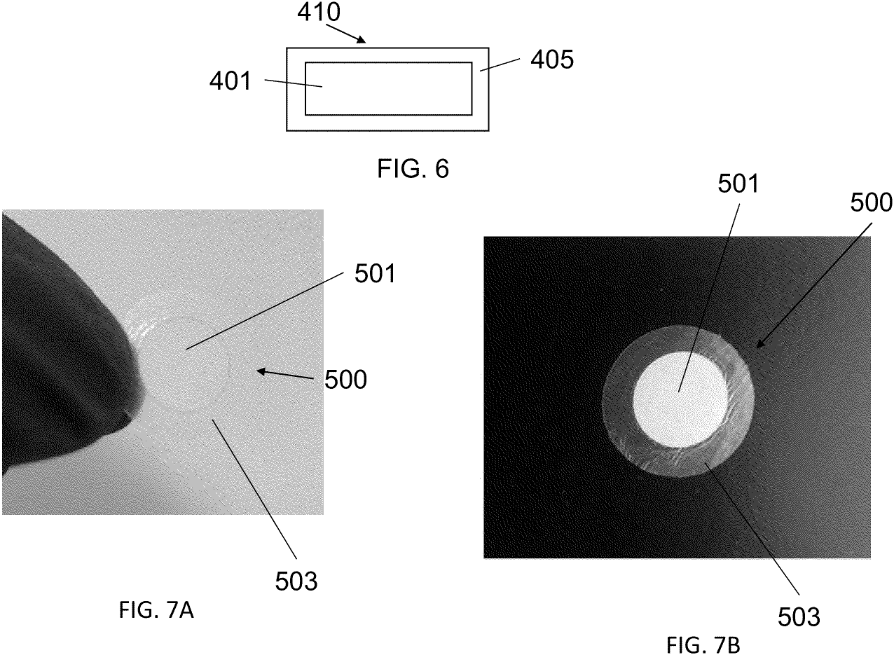

With reference to FIG. 5, the electrospun sheet from FIGS. 4A and 4B may be cut into pieces 401 and a laminate 403 applied to encase the pieces forming a sheet of laminated pieces 400. As depicted in FIG. 5, rectangular pieces 401 of electrospun material are of substantially uniform size and positioned with a sealed laminate border between adjacent pieces. With reference to FIG. 6, a bandage 410 is formed after one of the laminated pieces 401 and a sealed laminate border 405 is cut from the sheet of laminated pieces 400. Bandage 410 is fully encased in laminate material such the top and bottom surfaces are covered in laminate material and the sealed border covers the sides of the electrospun piece.

An embodiment of ¹⁶⁵Ho-containing electrospun laminated bandage 500 prior to neutron-activation, is shown in FIGS. 7A and 7B. The bandage 500 shows a circular electrospun piece 501 enclosed in laminate material 503, with the laminate material 503 having a greater diameter than the electrospun piece and providing a sealed border. While bandage 500 shown is circular, the bandage 500 may be of any shape including, for example, squares, rectangles, ovals, or any other type of adhesive bandage shape.

The bandages depicted in FIGS. 6 - 7A may be more safely handled post activation because the electrospun fibers and electrospun nanofibers are encased within the laminate.

With reference to FIGS. 7A and 7B, when the bandages 500, weighing approximately 23 mg, were neutron-activated for 10 h at 7.5 × 10¹² neutrons/cm²·s and radioactivity assayed at NC State over five separate occasions, the average radioactivity immediately after 10 h activation was 9.9 ± 2.2 mCi (n = 5).

The process described with reference to FIG. 2, referred to the HoIG bandages. However, one skilled in the art would understand that a similar process applies to creating bandages from mats having iron garnet nanoparticles and particles containing activatable nuclides as listed in FIG. 1. In other embodiments, carriers other than iron garnet may be used, including: polymeric-based nanoparticles or particles, lipid-based nanoparticles or particles, silica-based nanoparticles or particles, and other carriers. Carriers other than iron garnet may contain activatable nuclides as listed in FIG. 1.

In other embodiments, laminated samples were neutron-activated for more than 10 h in a thermal neutron flux of approximately at 7.5 × 10¹² neutrons/cm²·s. Lower and higher thermal neutron fluxes (including up to 10¹³ neutrons/cm2. s) may also be used. Neutron-activation times may include, for example, approximately 1 h, 2 h, 4 h, 5 h, 10 h, 15 h, 20 h, 24 h, 48 h, 72 h and greater. These neutron-activation times apply to iron garnet nanoparticles and iron garnet particles having activatable holmium nuclides. These neutron-activation times may also apply to iron garnet nanoparticles and iron garnet particles having one or more of the activatable nuclides (e.g., holmium, dysprosium, lanthanum, praseodymium, samarium, and/or rhenium) contained within the iron garnet particles or iron garnet nanoparticles. A benefit to longer activation times is that since activation and radioactivity are proportional, there is flexibility by providing a decaying period to achieve a desired radioactivity.

The activation times may be, for example, applied to carriers other than iron garnet, including: polymeric-based nanoparticles or particles, lipid-based nanoparticles or particles, silica-based nanoparticles or particles, and other carriers. Such carriers may incorporate, for example, activatable nuclides as listed in FIG. 1.

In other embodiments, the thickness of nylon laminate may vary with the activatable nuclides used. For activatable nuclides with beta emissions, a thinner nylon laminating film enveloping the bandage provides greater beta emission permeability. A thicker nylon laminating film is suitable if it does not impede beta particle therapy.

In other embodiments, the cut pieces of electrospun polymer nanofibrous or fibrous patch, fabric, sheet, or mat may be laminated, whether prepared by needle electrospinning or needleless electrospinning. By fully laminating the pieces, the polyacrylonitrile nanofibers or PAN fibers are contained within the laminated enclosure and are prevented from interacting with the skin or the diseased or tumorous surface. By fully laminating the pieces, the nuclide infused carrier nanoparticles and particles are contained within the laminated enclosure and are prevented from interacting with the skin or the diseased or tumorous surface even when the nuclides are activated.

In some embodiments, the bandage may be treated with materials that are “radiosensitizers”. Radiosensitizer compounds are drugs that act in combination with radiation to produce improved response, usually by making DNA more susceptible to radiation, or extending the life of free radicals produced by the radiation. Such radiosensitizers may be, for example, applied to the exterior of the lamination sealed bandage or directly to the skin exhibiting the skin disease or the location of the tumor. For cancer therapy, the purpose is to selectively enhance the effects of the dose to the tumor.

The following description is for a method for creating a bandage formed of carrier particles or nanoparticles having radiotherapeutic nanoparticle or nanoparticles. HoIG is produced by drying an HoIG preparation, annealing the HoIG preparation and grinding the holmium garnet to break up agglomerates, forming a powder.

The next steps form the input material for electrospinning. A dimethylformamide (anhydrous, 99.8%) (DMF) and polyacrylonitrile (MW 150,000) (PAN) mixture is stirred and heated until a 14.29% weight/volume PAN/DMF concentration is formed. The powdered HoIG is mixed with DMF to form a suspension. The suspension is placed in an ice bath and sonicated.

The PAN/DMF mixture and the holmium iron garnet powder may at this point me be moved into a first clean room in which electrospinning is performed. Use of and moving the items to a first clean room is preferred but is not essential. The PAN/DMF mixture is added to the holmium garnet/DMF suspension and is mixed to form a mixture for loading into an electrospinning instrument. The mixture is added to a carriage of an electrospinning instrument, for example, a needleless roll-to-roll electrospinning instrument. The electrospinning instrument proceeds to spin the mixture into fiber onto a paper substrate to form a mat. The mat may be cut into pieces by, for example, a punch or a laser cutting tool. The pieces may be placed into containers. The container may be moved into a second clean room to aid in minimizing cross contamination of free HoIG and/or nanofiber materials from the outer surface of the pieces during lamination. Use of and moving the pieces to a second clean room is preferred but is not essential.

The next step is the lamination process. The pieces may be removed from the container and encased in a polymer, such as, for example, wear-resistant nylon. Other polymers may also be used, such as, for example, polyesters, polypropylene, or polyethylene.

The outer laminated surface may be cleaned to minimize or remove any HoIG and/or nanofiber materials from the surface. The pieces may be packaged for shipment.

The pieces may be removed from packaging or remain in the package for activation. The pieces may be irradiated/activated with neutron irradiation such that therapeutic levels of radiation are emitted by the radiotherapeutic nanoparticle. An adhesive may be added to a side of a piece.

For treatment, the adhesive side of the piece may be placed on an area of skin of a patient for treatment. The length of treatment and the number of treatments may depend on the nature of the skin disease or cancer.

The method described may be used to produce electrospun sheets with other combinations of carrier nanoparticles or carrier particles and nuclides listed herein. Furthermore, the laminate material may be used to fully enclose these variants of electrospun sheets to form an encased radiotherapeutic bandage.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description, but is not intended to be exhaustive or limited to in the form disclosed. Many modifications and variations will be apparent to those of ordinary skill in the art. The embodiment was chosen and described in order to best explain various aspects and the practical application, and to enable others of ordinary skill in the art to understand various embodiments with various modifications as are suited to the particular use contemplated.

While several aspects of the present invention have been described and depicted herein, alternative aspects may be effected by those skilled in the art to accomplish the same objectives. Accordingly, it is intended by the appended claims to cover all such alternative aspects as fall within the true spirit and scope of the invention.

What is claimed is:

1. A bandage comprising:
an electrospun sheet comprising:
a polyacrylonitrile nanofiber embedded with a carrier nanoparticle or a carrier particle comprising:
an activatable nuclide selected from the group consisting of yttrium-89, lanthanum-139, praseodymium-141, samarium-152, dysprosium-164, holmium-165, rhenium-185, rhenium-187, and any combination thereof; and
a laminate enclosure, enclosing the electrospun sheet; and
wherein the bandage has a distribution of the carrier nanoparticle or the carrier particle to emit a relatively uniform radiation across the surface area of the bandage after neutron-activation.

2. The bandage of claim 1, wherein the carrier nanoparticle or the carrier particle further comprises carbon-based materials.

3. The bandage of claim 1, wherein the carrier nanoparticle or the carrier particle further comprises lipid-based materials.

4. The bandage of claim 1, wherein the carrier nanoparticle or the carrier particle further comprises silica-based materials.

5. The bandage of claim 1, wherein the carrier nanoparticle or the carrier particle comprises a polymeric-based materials.

6. The bandage of claim 1, wherein the carrier nanoparticle or the carrier particle comprises a graphene-based material.

7. The bandage of claim 2, wherein the carbon-based material consists of mesoporous carbon.

8. The bandage of claim 4, wherein the silica-based material consists of mesoporous silica.

9. The bandage of claim 1, wherein the laminate is a polymer and fully encases the bandage.

10. The bandage of claim 9, wherein the polymer is selected from the group consisting of nylon, polyester, polypropylene, polyethylene, and combinations thereof.

11. The bandage of claim 10, wherein the laminate covers all surfaces of the bandage and provides a sealed border around the sides of the bandage.

12. The bandage of claim 11, wherein the bandage has an adhesive applied to one surface.

13. The bandage of claim 10, wherein the laminate is nylon and has a tensile strength of at least 77220 kPa.

14. The bandage of claim 13, wherein the laminate has a thickness equal to or less than 0.5 mm.

15. The bandage of claim 13, wherein the laminate has a thickness equal to or less than 25.4 µm.

16. The bandage of claim 1, wherein the polyacrylonitrile nanofibers have a diameter from about 100 nm to about 350 nm.

17. The bandage of claim 16, wherein the polyacrylonitrile nanofibers have a diameter from about 150 nm to about 250 nm.

18. The bandage of claim 1, wherein the nanoparticles or particles have diameters of about 15 nm to about 250 nm.

19. The bandage of claim 18, wherein the nanoparticles have diameters of about 15 nm to about 200 nm.

20. A bandage comprising:
an electrospun sheet comprising:
a polyacrylonitrile nanofiber embedded with an iron garnet nanoparticle or an iron garnet particle comprising:
an activatable nuclide selected from the group consisting of yttrium-89, lanthanum-139, praseodymium-141, samarium-152, dysprosium-164, holmium-165, rhenium-185, rhenium-187, and combinations thereof; and
a laminate enclosure, enclosing the electrospun sheet; and
wherein the bandage has a distribution of the iron garnet nanoparticle or the iron garnet particle to emit a relatively uniform radiation across the surface area of the bandage after neutron-activation.

21. The bandage of claim 20, wherein the laminate enclosure is a polymer fully encasing the bandage.

22. The bandage of claim 21, wherein the laminate is selected from the group consisting of nylon, polyester, polypropylene, polyethylene, and combinations thereof.

23. The bandage of claim 22, wherein the laminate covers all surfaces of the bandage and provides a sealed border around the sides of the bandage.

24. The bandage of claim 23, wherein the bandage has an adhesive applied to one surface.

25. The bandage of claim 22, wherein the laminate is nylon and has a tensile strength of at least 77220 kPa.

26. The bandage of claim 22, wherein the laminate has a thickness equal to or less than 0.5 mm.

27. The bandage of claim 22, wherein the laminate has a thickness equal to or less than 25.4 µm.

28. The bandage of claim 20, wherein the polyacrylonitrile nanofiber or polyacrylonitrile fiber has a diameter from about 100 nm to about 350 nm.

29. The bandage of claim 28, wherein the polyacrylonitrile nanofiber or polyacrylonitrile fiber has a diameter from about 150 nm to about 250 nm.

30. The bandage of claim 20, wherein the nanoparticles or particles have diameters of about 15 nm to about 250 nm.

31. The bandage of claim 30, wherein the nanoparticles or particles have diameters of about 15 nm to about 200 nm.

32. The bandage of claim 20, wherein the activatable nuclide is holmium-165.

33. A method of forming radiotherapeutic bandages comprising:
a. preparing carrier nanoparticles or carrier particles comprising iron garnet and an activatable nuclide selected from the group consisting of yttrium-89, lanthanum-139, praseodymium-141, samarium-152, dysprosium-164, holmium-165, rhenium-185, rhenium-187, and combinations thereof;
b. drying and annealing the preparation;
c. grinding the preparation;
d. stirring and heating dimethylformamide (DMF) and polyacrylonitrile (PAN) and forming a concentration of 10% - 20% weight/volume PAN/DMF;
e. mixing the preparation and DMF to form a suspension;
f. sonicating the suspension in an ice bath;
g. adding PAN/DMF to the suspension;
h. mixing PAN/DMF and the suspension to form a mixture;
i. adding the mixture to a carriage of an electrospinning instrument;
j. electrospinning the mixture onto a paper substrate and forming a sheet;
k. cutting the sheet into pieces;
l. enclosing and sealing the pieces with a polymer laminate; and
m. forming a bandage by cutting or punching the enclosed and sealed laminated pieces.

34. The method of claim 33, wherein steps a. - f. are performed in a first space.

35. The method of claim 34, wherein after step f., moving the PAN/DMF and the carrier particle/DMF suspension to a first clean room and performing steps g. - k.

36. The method of claim 35, wherein after step k., placing the pieces into a clean container, moving to a second clean room and performing step 1.

37. The method of claim 35, wherein a clean down is performed after each of steps g. - k.

38. The method of claim 36, wherein a clean down is performed after step 1.

39. The method of claim 33, further comprises activating the bandage using neutron-activation.

40. The method of claim 39, further comprises adding an adhesive to one side of the bandage.

41. The method of claim 33, wherein stirring and heating the PAN/DMF concentration to make a 14.29% weight/volume PAN/DMF.

42. The method of claim 41, wherein mixing 1.21 g holmium iron garnet with 3.43 g DMF to form a suspension.

43. The method of claim 42, wherein sonicating the suspension to form a homogeneous suspension.
